(19) United States
(12) Patent Application Publication
(10) Pub. No.: US 2025/0064969 A1
(43) Pub. Date: Feb. 27, 2025

(54) METHOD FOR TREATING AUTOIMMUNE DISEASE

(71) Applicant: Suntec Medical, Inc., Walnut, CA (US)

(72) Inventors: Chun-Ting Cheng, Taipei (TW); Yuan-Chung Tsai, Taipei (TW); Pauline Ying Lau, Walnut, CA (US)

(21) Appl. No.: 18/940,614

(22) Filed: Nov. 7, 2024

Related U.S. Application Data
(63) Continuation of application No. PCT/US2023/022082, filed on May 12, 2023.
(60) Provisional application No. 63/342,381, filed on May 16, 2022.

(51) Int. Cl. A61K 47/69; A61K 9/107; A61K 47/54; A61K 47/60; C07K 16/28; C07K 16/32

(52) U.S. Cl. CPC A61K 47/6907; A61K 9/1075; A61K 47/545; A61K 47/60; C07K 16/2809; C07K 16/32

(57) ABSTRACT

The present invention provides a method of treating an autoimmune disease including type 1 diabetes, inflammatory bowel disease, rheumatoid arthritis. multiple sclerosis, systemic lupus erythematosus, psoriasis, myasthenia Gravis, and Hashimoto's thyroiditis. The method comprises the step of administering to a subject in need thereof an effective amount of micelles having an outer shell comprising one or more polymer-flavonoid conjugates, optionally an inner shell comprising one or more flavonoid oligomer, and a drug encapsulated within the shells. The present invention also provides a method of treating an autoimmune disease by administering to a subject in need thereof an effective amount of one or more polymer-flavonoid conjugate or one or more flavonoid oligomers.

### CROSS-REFERENCE TO RELATED APPLICATION(S)

This application is a continuation of PCT/US2023/022082, filed May 12, 2023; which claims the benefit of U.S. Provisional Application No. 63/342,381, filed May 16, 2022. The contents of the above-identified applications are incorporated herein by reference in their entireties.

### FIELD OF THE INVENTION

The present invention provides methods of treating autoimmune diseases. The method comprises the step of administering to a subject in need thereof an effective amount of (i) a PEG-flavonoid conjugate, (ii) a flavonoid oligomer, or (iii) micelles having a shell formed by one or more PEG-flavonoid conjugates or one or more flavonoid oligomers, or the combination thereof, and having an active agent encapsulated within the shell.

### BACKGROUND OF THE INVENTION

Autoimmune disease is a condition arising from an abnormal immune response to a functioning body part. Common autoimmune diseases include celiac disease, diabetes mellitus type 1, graves' disease, inflammatory bowel disease, multiple sclerosis, alopecia areata, Addison's disease, pernicious anemia, psoriasis, rheumatoid arthritis, systemic lupus erythematosus, myasthenia gravis, Hashimoto's thyroiditis, Vitiligo, Sjögren's syndrome, myositis, chronic inflammatory demyelinating polyneuropathy (CIDP), dermatomyositis, Guillain-Barre syndrome, psoriatic arthritis, ulcerative colitis and vasculitis.

### Rheumatoid Arthritis

Rheumatoid arthritis (RA) is a long-term autoimmune disorder that primarily affects joints. It typically results in warm, swollen, and painful joints. While the cause of rheumatoid arthritis is not clear, it is believed to involve a combination of genetic and environmental factors. The underlying mechanism involves the body's immune system attacking the joints. This results in inflammation and thickening of the joint capsule.

### Inflammatory Bowel Disease

Inflammatory bowel disease (IBD) is a group of inflammatory conditions of the colon and small intestine, Crohn's disease and ulcerative colitis being the principal types. Crohn's disease affects the small intestine and large intestine, as well as the mouth, esophagus, stomach and the anus, whereas ulcerative colitis primarily affects the colon and the rectum. In spite of Crohn's and UC being very different diseases, both may present with any of the following symptoms: abdominal pain, diarrhea, rectal bleeding, severe internal cramps/muscle spasms in the region of the pelvis and weight loss. Anemia is the most prevalent extraintestinal complication of inflammatory bowel disease. Associated complaints or diseases include arthritis, pyoderma gangrenosum, primary sclerosing cholangitis, and non-thyroidal illness syndrome (NTIS).

### Multiple Sclerosis

Multiple sclerosis (MS), also known as encephalomyelitis disseminata, is the most common demyelinating disease, in which the insulating covers of nerve cells in the brain and spinal cord are damaged. This damage disrupts the ability of parts of the nervous system to transmit signals, resulting in a range of signs and symptoms, including physical, mental, and sometimes psychiatric problems. Specific symptoms can include double vision, blindness in one eye, muscle weakness, and trouble with sensation or coordination. MS takes several forms, with new symptoms either occurring in isolated attacks (relapsing forms) or building up over time (progressive forms). Between attacks, symptoms may disappear completely, although permanent neurological problems often remain, especially as the disease advances. While the cause is unclear, the underlying mechanism is thought to be either destruction by the immune system or failure of the myelin-producing cells.

### Type 1 Diabetes

Type 1 diabetes (T1D), formerly known as juvenile diabetes, is an autoimmune disease that originates when very little or no insulin is produced by the islets of Langerhans (containing beta cells) in the pancreas. Insulin is a hormone required for the cells to use blood sugar for energy and it helps regulate normal glucose levels in the bloodstream. Before treatment this results in high blood sugar levels in the body. The common symptoms of this elevated blood sugar are frequent urination, increased thirst, increased hunger, weight loss, and other serious complications. Additional symptoms may include blurry vision, tiredness, and slow wound healing. Symptoms typically develop over a short period of time, often a matter of weeks. The cause of type 1 diabetes is unknown, but it is believed to involve a combination of genetic and environmental factors. The underlying mechanism involves an autoimmune destruction of the insulin-producing beta cells in the pancreas.

### Systemic Lupus Erythematosus

Systemic lupus erythematosus (SLE) is an autoimmune disease in which the body's immune system mistakenly attacks healthy tissue in many parts of the body. Symptoms vary among people and may be mild to severe. Common symptoms include painful and swollen joints, fever, chest pain, hair loss, mouth ulcers, swollen lymph nodes, feeling tired, and a red rash which is most commonly on the face. Often there are periods of illness, called flares, and periods of remission during which there are few symptoms. Systemic lupus erythematosus affects multiple organ systems and is characterized by a widespread loss of immune tolerance.

### Psoriasis

Psoriasis is a long-lasting, noncontagious autoimmune disease characterized by raised areas of abnormal skin. These areas are red, or purple on some people with darker skin, dry, itchy, and scaly. Psoriasis varies in severity from small, localized patches to complete body coverage. Injury to the skin can trigger psoriatic skin changes at that spot, which is known as the Koebner phenomenon. The cause of psoriasis is not fully understood. Currently, no cure is available for psoriasis but there are some treatment options, topical agents are typically used for mild disease, phototherapy for moderate disease, and systemic agents for severe disease. Flavonoids have the general structure of a 15-carbon skeleton, which consists of two phenyl rings (A and B) and a heterocyclic ring (C, the ring containing the embedded oxygen).

### Typical Treatments

Treatment depends on the type and severity of the condition. The majority of the autoimmune diseases are chronic and there is no definitive cure, but symptoms can be alleviated and controlled with treatment. Overall, the aim of the various treatment methods is to lessen the presented symptoms for relief and manipulate the body's autoimmune response, while still preserving the ability of the patient to combat diseases that they may encounter. Traditional treatment options may include immunosuppressant drugs to weaken the overall immune response, such as: non-steroidal anti-inflammatory drugs (NSAIDs) to reduce inflammation, glucocorticoids to reduce inflammation, and disease-modifying anti-rheumatic drugs (DMARDs) to decrease the damaging tissue and organ effects of the inflammatory autoimmune response.

### Flavonoids

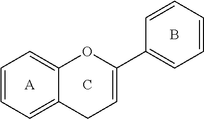

This carbon structure can be abbreviated C6-C3-C6. According to the IUPAC nomenclature, flavonoids can be classified into:
flavonoids or bioflavonoids
isoflavonoids, derived from 3-phenylchromen-4-one (3-phenyl-1,4-benzopyrone) structure
neoflavonoids, derived from 4-phenylcoumarine (4-phenyl-1,2-benzopyrone) structure

### BRIEF DESCRIPTION OF THE DRAWINGS

FIG. 1 illustrates one embodiment of a MINC (Multi-pathway Immune-modulating Nanocomplex Combination therapy)-agent, which is a micelle having a polymer-flavonoid conjugate, for example, a PEG-EGCG conjugate, in a shell and having an agent encapsulated.

FIG. 2 illustrates another embodiment of a MINC-agent, which is a micelle comprises a polymer-flavonoid conjugate, e.g., a PEG-EGCG conjugate in an outer shell and a flavonoid oligomer, for example, oligomeric EGCG (OEGCG), in an inner shell, and having an agent encapsulated.

FIG. 3 shows that OEGCG and PEG-EGCG inhibit LPS-induced inflammatory cytokine, TNF-α production in rheumatoid arthritis synovial fibroblast.

FIG. 4 shows that OEGCG and PEG-EGCG reduce inflammatory cytokine gene IL-6 and COX2 production in an inflammatory bowel disease cell model.

FIG. 5 shows that MINC-anti-CD3 reduces toxicity compared with anti-CD3.

FIG. 6 shows that different flavonoids in polymer-flavonoid conjugates and different flavonoid oligomers successfully generate MINC-anti-HER2 micelles with particle size around 100 nm.

FIG. 7. shows that different polymers in polymer-flavonoid conjugates successfully generate MINC-BSA micelles with particle size around 100 nm.

### DETAILED DESCRIPTION OF THE INVENTION

### Definitions

The term “about” is defined as ±10%, preferably ±5%, of the recited value.

The term “epigallocatechin gallate” refers to an ester of epigallocatechin and gallic acid, and is used interchangeably with “epigallocatechin-3-gallate” or EGCG.

The term “oligomeric EGCG” (OEGCG) refers to 3-30 monomers, preferably 2-20 monomers of EGCG that are covalently linked. OEGCG preferably contains 4 to 12 monomers of EGCG.

The term “polyethylene glycol-epigallocatechin gallate conjugate” or “PEG-EGCG refers to polyethylene glycol (PEG) conjugated to one or two molecules of EGCG. The term “PEG-EGCG” refer to both PEG-mEGCG conjugate (monomeric EGCG) and PEG-dEGCG (dimeric EGCG) conjugate.

The present invention provides methods of treating autoimmune diseases. The method comprises the step of administering to a subject in need thereof an effective amount of (i) a polymer-flavonoid conjugate, (ii) a flavonoid oligomer, or (iii) micelles having an outer shell formed by one or more polymer-flavonoid conjugates and optionally an inner shell formed by one or more flavonoid oligomer and a drug encapsulated within the shells.

### Flavonoids

Flavonoids suitable for the present invention have the general structure of Formula I:

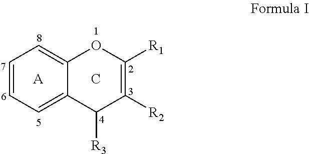

wherein:
R₁ is H, or phenyl;
R₂ is H, OH, Gallate, or phenyl; wherein the phenyl is optionally substituted by one or more (e.g., 2-3) hydroxyl;
R₃ is H, OH, or =O (oxo); or
R₁ and R₂ together form a close-looped ring structure; or
R₂ and R₃ together form close-looped ring structure.

The 2, 3, 4, 5, 6, 7, or 8 position of Formula I, can be linked to a group containing hydrocarbon, halogen, oxygen, nitrogen, sulfur, phosphorus, boron or metals.

Examples of flavonoids of Formula I include:

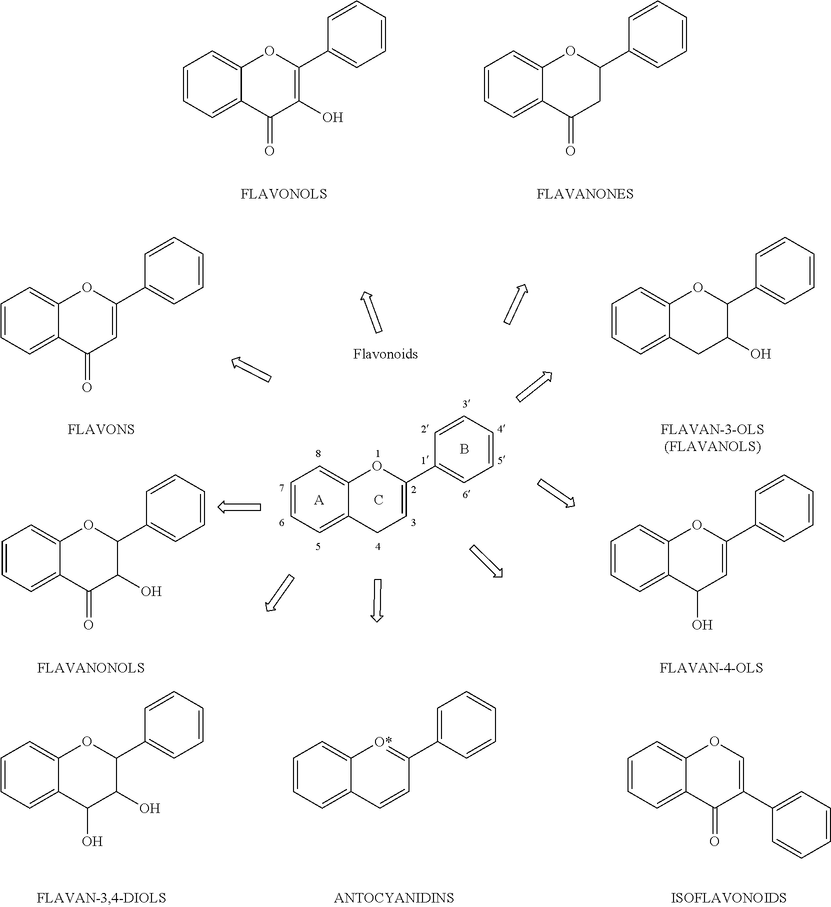

Preferred flavonoid compounds of Formula I include:

EGCG (CAS #989-51-5), EC (CAS #490-46-0), EGC (CAS #970-74-1) or ECG (CAS #1257-08-5).

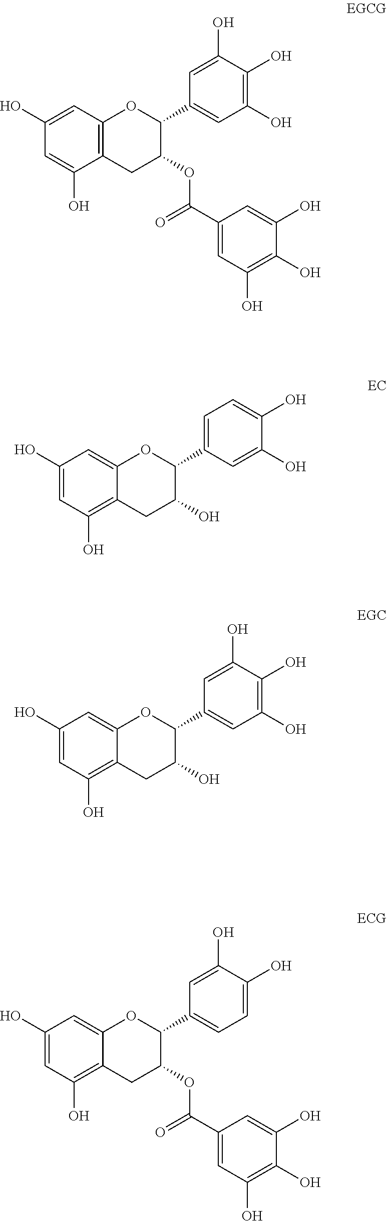 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]

### Polymer-Flavonoid Conjugate

A polymer-flavonoid conjugate, as used herein throughout the application, refers to a conjugate of a hydrophilic polymer and the flavonoid compound of Formula I.

A hydrophilic polymer refers to a polymer that is soluble in polar solvents and can form hydrogen bonds. Hydrophilic polymers suitable for the present polymer-flavonoid conjugates include, but not limited to: poly(ethylene glycol), aldehyde-derivatized hyaluronic acid, hyaluronic acid, dextran, diethylacetal conjugate (e.g. diethylacetal PEG), D-alpha-tocopheryl polyethylene glycol succinate, aldehyde-derivatized hyaluronic acid-tyramine, hyaluronic acid-aminoacetylaldehyde diethylacetal conjugate-tyramine, cyclotriphosphazene core phenoxymethyl(methylhydrazono)dendrimer or thiophosphoryl core phenoxymethyl(methylhydrazono)dendrimer. acrylamides, oxazolines, imines, acrylic acids, methacrylates, diols, oxiranes, alcohols, amines, anhydrides, esters, lactones, terephthalate, amides and ethers polyacrylamide, poloxamers, poly(N-isopropylacrylamide), poly(oxazoline), polyethylenimine, poly(acrylic acid), polymethacrylate, poly(ethylene glycol), poly(ethylene oxide), poly(vinylalcohol), poly(vinylpyrrolidinone), polyethers, poly(allylamine), polyanhydrides, poly(β-amino ester), poly(butylene succinate), polycaprolactone, polycarbonate, polydioxanone, poly(glycerol), polyglycolic acid, poly(3-hydroxypropionic acid), poly(2-hydroxyethyl methacrylate), poly(N-(2 hydroxypropyl)methacrylamide), polylactic acid, poly(lactic-co-glycolic acid), poly(ortho esters), poly(2 oxazoline), poly(sebacic acid), poly(terephthalate-co-phosphate), povidone and copolymers.

Preferred hydrophilic polymers include poly(ethylene glycol), hyaluronic acid, dextran, polyethylenimine, poloxamers, povidone, D-alpha-tocopheryl and polyethylene glycol succinate. The molecular weight of the hydrophilic polymer in the polymer-flavonoid conjugate is in general 1K-100K Daltons, preferably 2K-40K, 2K-50K, 2K-80K, 3K-80K, or 5K-40K Daltons.

In one embodiment, the polymer contains an aldehyde group which is conjugated to the 5, 6, 7, or 8 position (preferably 6 or 8 position) of the A ring of the flavonoid compound. In another embodiment, the polymer contains a thiol group which is conjugated to R₁ or R₂ of the B-ring of a flavonoid (when R₁ or R₂ is —OH).

In one embodiment, the polymer-flavonoid conjugate is PEG-EGCG, which is PEG conjugated to one or two molecules of epigallocatechin gallate (EGCG). PEG-EGCG, for example, can be prepared by conjugating aldehyde-terminated PEG to EGCG by attachment of the PEG via reaction of the free aldehyde group with the 5, 6, 7, or 8 position (preferably 6 or 8 position) of Formula I. See WO2006/124000 and WO2009/054813. PEG-EGCG can also be prepared by conjugating thio-terminated PEG to EGCG by attachment of the PEG via reaction of the free thio group with the R1 or R2 of Formula I, wherein, R1 or R2 is a phenyl group. See WO2015/171079.

### Flavonoid Oligomers

A flavonoid oligomer is a conjugate of one flavonoid with one or more flavonoids. The flavonoid oligomer can contain the same flavonoid (a homo oligomer) or different flavonoids (a hetero oligomer). Flavonoid oligomers useful for the present invention in general have 2-50 or 2-20, preferably 4-12 flavonoids of one or mixed types.

In some embodiment, a flavonoid oligomer is oligomeric EGC (OEGCG), oligomer EC (OEC), oligomer EGC (OEGC), or oligomer ECG (OECG). OEGCG refers to 3-20 monomers of EGCG that are covalently linked. OEGCG, for example, can be synthesized at 5, 6, 7, or 8 position (preferably 6 or 8 position) of the A ring according to WO2006/124000.

Because A-ring is present in all of the flavonoids according to Formula 1, other oligomeric flavonoids can be made similarly according to WO2006/124000. For example, OEC, OEGC, and OECG can also be made according to WO2006/124000.

### MINC-Agent

MINC (Multi-pathway Immune-modulating Nanocomplex Combination therapy) is a platform technology, utilizing the bioactivity of polymer-flavonoid conjugates or flavonoid oligomers that form micelles in a solution.

MINC platform can encapsulate additional agents to form a nanoparticle composition for a combination therapy. MINC-agent is a micelle with a shell formed by one or more polymer—flavonoid conjugates or one or more flavonoid oligomers, or the combination thereof, and has an agent encapsulated within the shell. The agent, as used herein, referred to a molecule that have a therapeutic activity to treat an autoimmune disease including but not limited to an anti-inflammatory agent such as an anti-inflammatory antibody, an anti-inflammatory cytokine, an anti-inflammatory compound, or an antibody, or a compound.

MINC platform can reduce the toxicity of the encapsulated agent, which expands the effective dosage window of the agent in an autoimmune disease. For example, anti-CD3 is a broad-spectrum immune suppressor for treating various autoimmune diseases. However, anti-CD3 has high toxicity that restricts its clinical use. MINC-anti-CD3 formulation reduces the toxicity of anti-CD3. MINC-anti-CD3 is a safe drug for the treatment of autoimmune diseases.

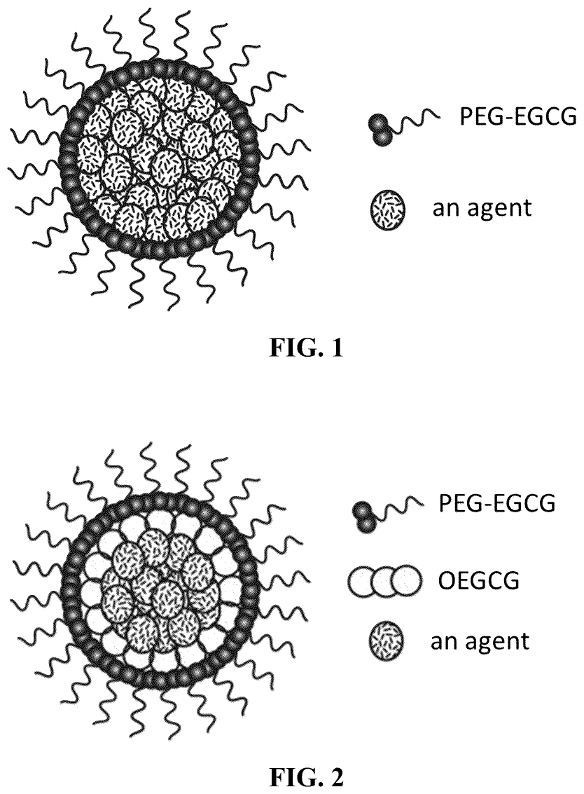

In one embodiment, MINC-agent is a micelle comprises a polymer-flavonoid conjugate, for example, a PEG-EGCG conjugate, in a shell and with an agent encapsulated (FIG. 1).

In another embodiment, MINC-agent is a micelle comprises a polymer-flavonoid conjugate, for example, a PEG-EGCG conjugate in an outer shell and a flavonoid oligomer, for example, oligomeric EGCG (OEGCG), in an inner shell, with an agent encapsulated (FIG. 2).

When the agent is a drug, the MINC-Agent composition comprises two or more components that have therapeutic activities, which are complementary in function to form a multiple targeted combination therapy by its backbone components (a flavonoid conjugate or a flavonoid oligomer), and the encapsulated drug. The MINC-Agent is stable in a hydrophilic environment, such as blood circulation.

In one embodiment, the agent in MINC-agent is an anti-inflammatory antibody include but not limited to anti-IL-1, anti-IL-1α, anti-IL-1β, anti-IL-1R, anti-IL-2, anti-IL-6, anti-IL-6R, anti-IL-7, anti-IL-7R, anti-IL-10, anti-IL-11, anti-IL-12, anti-IL-17, anti-IL-18, anti-IL-23, anti-IFN-α, anti-IFN-β, anti-IFN-γ, anti-TNF-α, anti-CD3, anti-CD20, anti-TGF-β, anti-T lymphocyte globulin (ATG). Such MINC-Agent is suitable to treat rheumatoid arthritis, inflammatory bowel disease, multiple sclerosis, type 1 diabetes, systemic lupus erythematosus, and psoriasis.

In one embodiment, the agent in MINC-agent is an anti-inflammatory antibody include but not limited to Adalimumab, Infliximab, Golimumab, Rituximab, Certolizumab, Tocilizumab, Sarilumab, Vedolizumab, Etrolizumab, Natalizumab, PF-00547659, Ustekinumab, Alemtuzumab, Daclizumab, Ocrelizumab, Canakinumab, Dapirolizumab, Secukinumab, Ixekizumab. Such MINC-Agent is suitable to treat rheumatoid arthritis, inflammatory bowel disease, multiple sclerosis, type 1 diabetes, systemic lupus erythematosus, and psoriasis.

In one embodiment, the agent in MINC-agent is an antibody including but not limited to Adalimumab, Infliximab, Golimumab, Rituximab, Certolizumab, Tocilizumab, Sarilumab Such MINC-Agent is suitable to treat rheumatoid arthritis.

In one embodiment, the agent in MINC-agent is an antibody including but not limited to Vedolizumab, Etrolizumab, Natalizumab, PF-00547659, Ustekinumab, Golimumab, Adalimumab, Certolizumab, Natalizumab, Infliximab. Such MINC-Agent is suitable to treat inflammatory bowel disease.

In one embodiment, the agent in MINC-agent is an antibody including but not limited to Natalizumab, Alemtuzumab, Daclizumab, Ocrelizumab. Such MINC-Agent is suitable to treat multiple sclerosis.

In one embodiment, the agent in MINC-agent is an antibody including but not limited to Canakinumab, Ustekinumab, Rituximab, Otelixizumab, Teplizumab, visilizumab. Such MINC-Agent is suitable to treat type 1 diabetes.

In one embodiment, the agent in MINC-agent is an antibody including but not limited to Dapirolizumab, Belimumab, Anifrolumab, BIIB059. Such MINC-Agent is suitable to treat systemic lupus erythematosus.

In one embodiment, the agent in MINC-agent is an antibody including but not limited to Infliximab, Adalimumab, Ustekinumab, Secukinumab, Ixekizumab. Such MINC-Agent is suitable to treat psoriasis.

In one embodiment, the agent in MINC-agent is an anti-inflammatory compound including but not limited to Leflunomide, Aspirin, Naproxen, Diclofenac, Ibuprofen, Methotrexate, Sulphasalazine, Hydroxychloroquine, Sulfasalazine, Baricitinib, Tofacitinib, Azathioprine, Cyclosporine, Minocycline, Ozanimod, Mesalamine, Olsalazine, Balsalazide, Prednisone, Hydrocortisone, Budesonide, 6-mercaptopurine, Methotrexate, Metronidazole, Glatiramer acetate, Fingolimod, Teriflunomide, Dimethyl fumarate, Laquinimod, Mitoxantrone, Dalfampridine, Metformin, Tolbutamide, Chlorpropamide, Tolazamide, Glyburide, Glipizide, Glimepiride, Repaglinide, Nateglinide, Sitagliptin, Saxagliptin, Linagliptin, Alogliptin, Rosiglitazone, Pioglitazone, Acarbose, Miglitol, Canagliflozin, Dapagliflozin, Empagliflozin, BT-063, Fenebrutinib, Iberdomide, Cenerimod, Evobrutinib, Bortezomib, Ibuprofen, Naproxen, Chloroquine, Quinacrine, Betamethasone dipropionate, prednisolone, Methylprednisolone sodium succinate, Cyclophosphamide, Mycophenolate mofetil, Prasterone, Dehydroepiandrosterone, Apremilast, Ruxolitinib, Baricitinib, ASP015K, Sotrastaurin, Fostamatinib, Tamatinib. Such MINC-Agent is suitable to treat rheumatoid arthritis, inflammatory bowel disease, multiple sclerosis, type 1 diabetes, systemic lupus erythematosus, and psoriasis.

In one embodiment, the agent in MINC-agent is a compound including but not limited to Leflunomide, Aspirin, Naproxen, Diclofenac, Ibuprofen, Methotrexate, Sulphasalazine, Hydroxychloroquine, Sulfasalazine, Baricitinib, Tofacitinib, Azathioprine, Cyclosporine, Minocycline. Such MINC-Agent is suitable to treat rheumatoid arthritis.

In one embodiment, the agent in MINC-agent is a compound including but not limited to Tofacitinib, Ozanimod, Sulfasalazine, Mesalamine, Olsalazine, Balsalazide, Prednisone, Hydrocortisone, Budesonide, Azathioprine, 6-mercaptopurine, Methotrexate, Cyclosporine, Metronidazole, Methylpred-nisolone. Such MINC-Agent is suitable to treat inflammatory bowel disease.

In one embodiment, the agent in MINC-agent is a compound including but not limited to Glatiramer acetate, Fingolimod, Teriflunomide, Dimethyl fumarate, Laquinimod, Mitoxantrone, Dalfampridine. Such MINC-Agent is suitable to treat multiple sclerosis.

In one embodiment, the agent in MINC-agent is a compound drug including but not limited to Metformin, Tolbutamide, Chlorpropamide, Tolazamide, Glyburide, Glipizide, Glimepiride, Repaglinide, Nateglinide, Sitagliptin, Saxagliptin, Linagliptin, Alogliptin, Rosiglitazone, Pioglitazone, Acarbose, Miglitol, Canagliflozin, Dapagliflozin, Empagliflozin. Such MINC-Agent is suitable to treat type 1 diabetes.

In one embodiment, the agent in MINC-agent is a compound including but not limited to Hydroxychloroquine, Prednisone, Methylprednisolone, Cyclophosphamide, Azathioprine, Mycophenolate, BT-063, Fenebrutinib, Iberdomide, Cenerimod, Evobrutinib, Bortezomib, Ibuprofen, Naproxen, Aspirin, Chloroquine, Quinacrine, Betamethasone dipropionate, Prednisone prednisolone, Methylprednisolone sodium succinate, Cyclophosphamide, Mycophenolate mofetil, Cyclosporine, Methotrexate, Prasterone, Dehydroepiandrosterone. Such MINC-Agent is suitable to treat systemic lupus erythematosus.

In one embodiment, the agent in MINC-agent is a compound including but not limited to Apremilast, Tofacitinib, Ruxolitinib, Baricitinib, ASP015K, Sotrastaurin, BMS-582949, Fostamatinib, Tamatinib. Such MINC-Agent is suitable to treat psoriasis.

In one embodiment, the agent in MINC-agent is an anti-inflammatory protein including but not limited to Etanercept, Abatacept, Mongersen, Exenatide, Liraglutide, Albiglutide, Dulaglutide, Abatacept, Alefacept, IL-2, GCSF, RSLV-132, RC18, AMG592. Such MINC-Agent is suitable to treat rheumatoid arthritis, inflammatory bowel disease, multiple sclerosis, type 1 diabetes, systemic lupus erythematosus, and psoriasis.

For example, the MINC-agent is anti-IL-1 encapsulated in the micelle formed by PEG-EGCG and a flavonoid oligomer OEGCG. See WO2009/054813 for the structure and formulation method.

For example, the MINC-agent is etanercept encapsulated in the micelle formed by PEG-EGCG and a flavonoid oligomer OEGCG. See WO2009/054813 for the structure and formulation method.

For example, the MINC-agent is leflunomide encapsulated in the micelle formed by a PEG-EGCG. See WO2011/112156 for the structure and formulation method.

### Pharmaceutical Compositions

The present invention uses pharmaceutical compositions comprising the hydrophilic polymer-flavonoid conjugate, flavonoid oligomer, or MINC-agent composition as described in the application, and optionally one or more pharmaceutically acceptable carriers. The nanoparticle composition in a pharmaceutical composition in general is about 1-90%, preferably 20-90%, or 30-80% for a tablet, powder, or parenteral formulation. The PEG-flavonoid conjugate, flavonoid oligomer, or MINC-agent composition in a pharmaceutical composition in general is 1-100%, preferably 20-100%, 50-100%, or 70-100% for a capsule formulation. The nanoparticle composition in a pharmaceutical composition in general is 1-50%, 5-50%, or 10-40% for a liquid suspension formulation.

In one embodiment, the pharmaceutical composition can be in a dosage form such as tablets, capsules, granules, fine granules, powders, suspension, patch, parenteral, injectable, or the like. The above pharmaceutical compositions can be prepared by conventional methods.

Pharmaceutically acceptable carriers, which are inactive ingredients, can be selected by those skilled in the art using conventional criteria. The pharmaceutically acceptable carriers may contain ingredients that include, but are not limited to, saline and aqueous electrolyte solutions; ionic and nonionic osmotic agents, such as sodium chloride, potassium chloride, glycerol, and dextrose; pH adjusters and buffers, such as salts of hydroxide, phosphate, citrate, acetate, borate, and trolamine; antioxidants, such as salts, acids, and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants, such as lecithin and phospholipids, including, but not limited to, phosphatidylcholine, phosphatidylethanolamine and phosphatidyl inositol; poloxamers and poloxamines; polysorbates, such as polysorbate 80, polysorbate 60, and polysorbate 20; polyethers, such as polyethylene glycols and polypropylene glycols; polyvinyls, such as polyvinyl alcohol and polyvinylpyrrolidone (PVP, povidone); cellulose derivatives, such as methylcellulose, hydroxypropyl cellulose, hydroxyethyl cellulose, carboxymethyl cellulose, and hydroxypropyl methylcellulose and their salts; petroleum derivatives, such as mineral oil and white petrolatum; fats, such as lanolin, peanut oil, palm oil, and soybean oil; mono-, di-, and triglycerides; polysaccharides, such as dextrans; and glycosaminoglycans, such as sodium hyaluronate. Such pharmaceutically acceptable carriers may be preserved against bacterial contamination using well-known preservatives, which include, but are not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet, capsule, or parenteral formulation of the active compound may contain other excipients that have no bioactivity and no reaction with the active compound. Excipients of a tablet or a capsule may include fillers, binders, lubricants and glidants, disintegrators, wetting agents, and release rate modifiers. Examples of excipients of a tablet or a capsule include, but are not limited to, carboxymethylcellulose, cellulose, ethylcellulose, hydroxypropylmethylcellulose, methylcellulose, karaya gum, starch, tragacanth gum, gelatin, magnesium stearate, titanium dioxide, poly(acrylic acid), and polyvinylpyrrolidone.

For example, a tablet formulation may contain inactive ingredients, such as colloidal silicon dioxide, crospovidone, hypromellose, magnesium stearate, microcrystalline cellulose, polyethylene glycol, sodium starch glycolate, and titanium dioxide. A capsule formulation may contain inactive ingredients, such as gelatin, magnesium stearate, and titanium dioxide. A powder oral formulation may contain inactive ingredients, such as silica gel, sodium benzoate, sodium citrate, sucrose, and xanthan gum.

The pharmaceutical composition can be applied by local administration and systemic administration. Local administration includes topical administration. Systemic administration includes oral, parenteral (such as intravenous, intramuscular, subcutaneous, or rectal), and other systemic routes of administration. In systemic administration, the active compound first reaches plasma and then distributes into target tissues. Parenteral administration, such as intravenous bolus injection or intravenous infusion, and oral administration are preferred routes of administration.

### Method of Treatment

The present invention is directed to a method of preventing or treating an autoimmune disease such as rheumatoid arthritis, inflammatory bowel disease, multiple sclerosis, type 1 diabetes, systemic lupus erythematosus, and psoriasis.

### Polymer-Flavonoid

In the first aspect of the invention, the method comprises the step of administering an effective amount of a polymer-flavonoid conjugate to a subject in need thereof to treat an autoimmune disease. “An effective amount” as used in this application, is the amount effective to treat a disease by ameliorating the pathological condition or reducing the symptoms of the disease.

The polymer-flavonoid conjugate of the present invention has immune modulation and anti-inflammatory activity for treating autoimmune diseases.

In one embodiment, the flavonoid is EGCG, EC, EGC, or ECG.

In one embodiment, the polymer is a hydrophilic polymer having a molecular weight of 1,000 to 100,000 daltons, and is selected from the group consisting of: PEG, hyaluronic acid, dextran, polyethylenimine, poloxamers, povidone, D-alpha-tocopheryl, and polyethylene glycol succinate.

A preferred polymer-flavonoid conjugate is PEG-EGCG.

In one embodiment, the autoimmune disease is associated with joints and muscles and is rheumatoid arthritis, psoriatic arthritis, Sjögren's syndrome, or systemic lupus erythematosus.

In one embodiment, the autoimmune disease is associated with digestive tract and is inflammatory bowel disease, Celiac disease, or ulcerative colitis.

In one embodiment, the autoimmune disease is associated with endocrine system and is type 1 diabetes, Graves' disease, Hashimoto's thyroiditis, or Addison's disease.

In one embodiment, the autoimmune disease is associated with skin and is psoriasis, dermatomyositis, or Vitiligo.

In one embodiment, the autoimmune disease is associated with the nervous system and is multiple sclerosis, chronic inflammatory demyelinating polyneuropathy, or Guillain-Barre syndrome.

In one embodiment, the autoimmune disease is Myasthenia gravis, autoimmune vasculitis, pernicious anemia, vasculitis, or myositis.

Dosing for a polymer-flavonoid, e.g., PEG-EGGC, for injection, is in general 0.1-10000 mg/kg, 0.6-1200 mg/kg (total weight of the PEG-flavonoid/subject body weight), or 1-1000 mg/kg.

### Flavonoid Oligomer

In a second aspect of the invention, the method comprises the step of administering an effective amount of a flavonoid oligomer to a subject in need thereof. The flavonoid oligomer of the present invention has immune modulation and anti-inflammatory activity for treating autoimmune diseases.

In one embodiment, the flavonoid oligomer is an oligomer of EGCG, EC, EGC, or ECG.

In one embodiment, the flavonoid oligomer comprises 4-12 flavonoids of EGCG, EC, EGC, or ECG.

In one embodiment, the autoimmune disease is associated with joints and muscles and is rheumatoid arthritis, psoriatic arthritis, Sjögren's syndrome, or systemic lupus erythematosus.

In one embodiment, the autoimmune disease is associated with digestive tract and is inflammatory bowel disease, Celiac disease, or ulcerative colitis.

In one embodiment, the autoimmune disease is associated with endocrine system and is type 1 diabetes, Graves' disease, Hashimoto's thyroiditis, or Addison's disease.

In one embodiment, the autoimmune disease is associated with skin and is psoriasis, dermatomyositis, or Vitiligo.

In one embodiment, the autoimmune disease is associated with the nervous system and is multiple sclerosis, chronic inflammatory demyelinating polyneuropathy, or Guillain-Barre syndrome.

In one embodiment, the autoimmune disease is Myasthenia gravis, autoimmune vasculitis, pernicious anemia, vasculitis, or myositis.

Dosing for a flavonoid oligomer, e.g., OEGCG, for injection, is in general 0.1-1000 mg/kg, 0.1-100 mg/kg (total weight of the flavonoid oligomer/subject body weight), or 1-100 mg/kg.

### MINC-Drug

In a third aspect of the invention, the method comprises the step of administering to a subject in need thereof an effective amount of micelles having an outer shell comprising one or more polymer-flavonoid conjugates and optionally an inner shell comprising one or more flavonoid oligomer and a drug encapsulated within the shells, to treat an autoimmune disease. In one embodiment, the micelles comprise both an outer shell and an inner shell. In another embodiment, the micelles do not have an inner shell, and only have one shell comprising one or more polymer-flavonoid.

The polymer-flavonoid conjugate or the flavonoid oligomer provides its own therapeutic effect and further delivers said agent for treating autoimmune diseases.

In one embodiment, the polymer is a hydrophilic polymer having a molecular weight of 1,000 to 100,000 Daltons, and is selected from the group consisting of: poly(ethylene glycol) (PEG), hyaluronic acid, dextran, polyethylenimine, poloxamers, povidone, D-alpha-tocopheryl, and polyethylene glycol succinate;

In one embodiment, the flavonoid oligomer comprises 2-50 or 2-20 flavonoids of EGCG, EC, EGC, or ECG;

In one embodiment, the shell is formed by PEG-EGCG.

In one embodiment, the shell is formed by PEG-EGCG and OEGCG.

In one embodiment, the autoimmune disease is type 1 diabetes, and the drug is anti-CD3, anti-IL-1β, anti-IL-1R, GAD, HSP60, B9-23 peptide, MIP, IL-2, anti-CTLA4, anti-CD40, anti-CD20, anti-PD1, TKI, GLP-1, anti-STAT1, anti-S1P, anti-12/15-LOX, IL-35, HDAC inhibitor, anti-DYRK1A, NFAT, Substance P, LRH-1, IL-10, IGF, IGFBP1, ARX, GCSF, VMAT-2, or RAE-1.

In one embodiment, the autoimmune disease is inflammatory bowel disease, and the drug is anti-CD3, anti-ITGA4, anti-IL-12B, anti-TNF, anti-JAK2, anti-PTGS1/2, PPAR-γ, anti-ITGB7, NR3C1, anti-JAK3, anti-ALOX5, anti-TYK2, anti-PPAT, VDR, anti-MMP1, anti-MMP7, anti-DHFR, anti-MMP13, anti-ATP4A, S1PR1, anti-IL-36, anti-IL-6, anti-IL-6R, anti-TLR9, IL-10, anti-TGF-β, anti-IL-9, anti-IL-12, anti-smad7, anti-integrins, anti-MDR1, anti-PPAR-γ, anti-IL-35, anti-IL-27, or anti-IL17.

In one embodiment, the autoimmune disease is rheumatoid arthritis, and the drug is anti-TNF-α, anti-IL-1R, anti-IL-1, anti-IL-6R, anti-IL-6, anti-IL-2, IL-10, IL-15, IL-18, anti-IL-17, anti-IL-17R, anti-IFN-γ, anti-CCL2, anti-CCR9, anti-CX3CL1, anti-CCR1, anti-CCR2, anti-CCR5, anti-TLR4, anti-GRK2, anti-MEK, anti-MMP9, anti-CD3, anti-CD80, anti-BTK, anti-IL-23, anti-GM-CSF, anti-CXCL10, anti-CXCL12, anti-CXCL13, anti-CXCL16, anti-CXCR1/2, anti-CXCL3, anti-CXCR4, anti-CXCR7, anti-CCR7, anti-JAK, anti-p38 MAPK, anti-IRAK-4, anti-CD20, anti-CD11a, or anti-CD19.

In one embodiment, the autoimmune disease is multiple sclerosis, and the drug is ROS scavenger, anti-NF-κB, anti-NOX2, anti-iNOS, MDA, HNE, 4-HHR inhibitor, IFN-β, NRF2 activator, PGC-1α, anti-IL-1β, anti-TNF-α, anti-IL-6, anti-IL-6R, anti-MMP-9, anti-IL-12, anti-IFN-γ, CNTF, anti-caspase 3, anti-MIP-1α, anti-TLR2, anti-CD3, anti-ICAM-1, anti-CCR6, anti-IL2, anti-IL-9, anti-17, TGF-β, IL-4, or FoxP3 activator.

In one embodiment, the autoimmune disease is systemic lupus erythematosus, and the drug is anti-CD20, anti-CD19, anti-CD22, proteosome inhibitor, anti-BAFF, anti-BTK, ant-CD28, anti-CD40L, anti-CD40, anti-IL12, anti-IL23, anti-IL17, IL2, mTOR inhibitor, calcineurin inhibitor, anti-JAK, anti-IFN-α, anti-IFNAR, anti-IFN-γ, anti-TLR7, anti-TLR9, anti-TLR8, anti-CTLA4, anti-IL6, or anti-CD3.

In one embodiment, the autoimmune disease is psoriasis, and the drug is anti-CD3, anti-TNF, anti-IL-17A, anti-IL-12, anti-IL-23, anti-36, anti-PDE4, anti-JAK, anti-RORgT, anti-IRAK-4, anti-CCL20, anti-CXCL8, anti-IL-1β, anti-iNOS, anti-IL-8, anti-IFNγ, anti-S1PR1, anti-Tyk2, anti-A3 adenosine receptor, or mTOR inhibitor.

In one embodiment, the autoimmune disease is Myasthenia Gravis, and the drug is Anti-CD3, Anti-FcRN, anti-CD20, amti-CD19, anti-CD154, proteasome inhibitor, anti-CD38, anti-CD40, anti-IL6, anti-TNF, anti-BARF, anti-BTK, anti-IL-6R, anti-IL-17A, anti-C5, or anti-AChR.

In one embodiment, the autoimmune disease is Hashimoto's thyroiditis, and the drug is anti-CD3, anti-TSHR, anti-TPO, anti-Tg, anti-NIS, anti-IL-12, anti-IFN-γ, anti-IL-4, anti-IL-5, anti-IL-10, anti-IL-17, anti-IL-6, anti-FLT3L, anti-LypR620W, anti-ICAM1, anti-IL17, anti-IL-23, anti-RHOH, anti-RNASET2, anti-SLAMF6, anti-TNF-α, or anti-FcRN.

Dosing of the MINC-agent is based on the known dosage of the agents for treating a particular disease and the subject condition. The dosage can be a Food and Drug Administration (FDA) approved dosage or a dosage used in clinical trial.

In MINC-agent, in general, the dosage of PEG-EGCG combined with OEGCG is between 10 μg/kg to 100 mg/kg.

The concentration for the encapsulated drug agents can be as low as 1 μg/kg (e. g., for cytokine drugs, G-CSF) and as high as 10 mg/kg (for antibody drugs, e.g., certolizumab is at this level).

For example, when MINC-agent comprises an antibody, including but not limited to anti-IL-1, anti-IL-1α, anti-IL-1β, anti-IL-1R, anti-IL-2, anti-IL-6, anti-IL-6R, anti-IL-7, anti-IL-7R, anti-IL-10, anti-IL-11, anti-IL-12, anti-IL-17, anti-IL-18, anti-IL-23, anti-IFN-α, anti-IFN-β, anti-IFN-γ, anti-TNF-α, anti-CD20, anti-TGF-β, anti-T lymphocyte globulin (ATG) for treating rheumatoid arthritis, multiple sclerosis, inflammatory bowel disease, type 1 diabetes, systemic lupus erythematosus, and psoriasis, the antibody can be administered at a range of 0.1-10 mg/kg IV once every one to four weeks.

For example, for treating rheumatoid arthritis, multiple sclerosis, inflammatory bowel disease, type 1 diabetes, systemic lupus erythematosus, psoriasis, or other autoimmune disease in an adult human, anti-TNF-α is administered 0.1-10 mg/kg IV once every one to four weeks. MINC-anti-TNF-α comprises same dose range of anti-TNF-α can be used for treating these diseases.

For example, for treating rheumatoid arthritis, multiple sclerosis, inflammatory bowel disease, type 1 diabetes, systemic lupus erythematosus, psoriasis or other autoimmune diseases in an adult human, etanercept is administered 0.1-10 mg/kg IV once every one to four weeks. MINC-etanercept comprises same dose range of etanercept can be used for treating these diseases.

For example, for treating rheumatoid arthritis, multiple sclerosis, inflammatory bowel disease, type 1 diabetes, systemic lupus erythematosus, and psoriasis in an adult human, methylprednisolone is administered 0.5-50 mg/kg IV once every one to four weeks. MINC-methylprednisolone comprises same dose range of methylprednisolone can be used for treating these diseases.

For example, when a MINC-agent comprises an anti-inflammatory compound, including but not limited to leflunomide, aspirin, naproxen, diclofenac, ibuprofen, methotrexate, sulphasalazine, hydroxychloroquine, sulfasalazine, baricitinib, tofacitinib, azathioprine, cyclosporine, minocycline, ozanimod, mesalamine, olsalazine, balsalazide, prednisone, hydrocortisone, budesonide, 6-mercaptopurine, metronidazole, methylpred-nisolone, glatiramer acetate, fingolimod, teriflunomide, dimethyl fumarate, laquinimod, mitoxantrone, dalfampridine, metformin, tolbutamide, chlorpropamide, tolazamide, glyburide, glipizide, glimepiride, repaglinide, nateglinide, sitagliptin, saxagliptin, linagliptin, alogliptin, rosiglitazone, pioglitazone, acarbose, miglitol, canagliflozin, dapagliflozin, empagliflozin, cyclophosphamide, mycophenolate, BT-063, fenebrutinib, iberdomide, cenerimod, evobrutinib, bortezomib, chloroquine, quinacrine, betamethasone dipropionate, prasterone, dehydroepiandrosterone, apremilast, ruxolitinib, ASP015K, sotrastaurin, BMS-582949, fostamatinib or tamatinib for treating rheumatoid arthritis, multiple sclerosis, inflammatory bowel disease, type 1 diabetes, systemic lupus erythematosus, and psoriasis, the anti-inflammatory compound can be administered at a range of 0.1-10 mg/kg IV once every one to four weeks.

For example, when a MINC-agent comprises a non-steroidal anti-inflammatory drug (NSAID), including but not limited to, ibuprofen, naproxen, diclofenac, celecoxib, mefenamic acid, etoricoxib, indomethacin or aspirin for treating rheumatoid arthritis, multiple sclerosis, inflammatory bowel disease, type 1 diabetes, systemic lupus erythematosus, and psoriasis, the anti-inflammatory drug can be administered at a range of 0.1-10 mg/kg IV once to twice daily.

The present invention is useful in treating human and non-human animals. For example, the present invention is useful in treating a mammal subject, such as humans, horses, pigs, cats, and dogs.

The following examples further illustrate the present invention. These examples are intended merely to be illustrative of the present invention and are not to be construed as being limiting.

### EXAMPLES

### Active Ingredients

### OEGCG:

OEGCG is oligomerized EGCG. OEGCG is prepared according to WO2006/124000.

### PEG-EGCG:

PEG-EGCG is PEG conjugated with one or two EGCG. PEG-EGCG is prepared according to WO2006/124000, WO2009/054813, or WO2015/171079.

### MINC-Agents:

MINC-Agents are prepared by encapsulated an agent within the micelle formed by PEG-EGCG and OEGCG, according to the method in WO2006/124000 or WO2009/054813.

### Example 1: OEGCG and PEG-EGCG Inhibit LPS-Induced Rheumatoid Arthritis Synovial Fibroblast Inflammatory Cytokine TNF-α Production

### Materials

TNF-alpha ELISA Kit, Human (Sino biological, Cat: KIT10602)

Rheumatoid arthritis synovial fibroblast (RASF) (from RIKEN)

### Method

Rheumatoid arthritis synovial fibroblast (RASF) cells were seeded at 1×10⁴ cells/well in 96 well plate and incubated overnight. On the second day, cells were pretreated with different concentrations of OEGCG or PEG-EGCG for 20 mins and then co-incubated with 1 g/mL of LPS for 48 hours. At the end of treatment, cell supernatant was collected and subjected to human TNF-α ELISA kit according to the manufacture instruction.

### Results

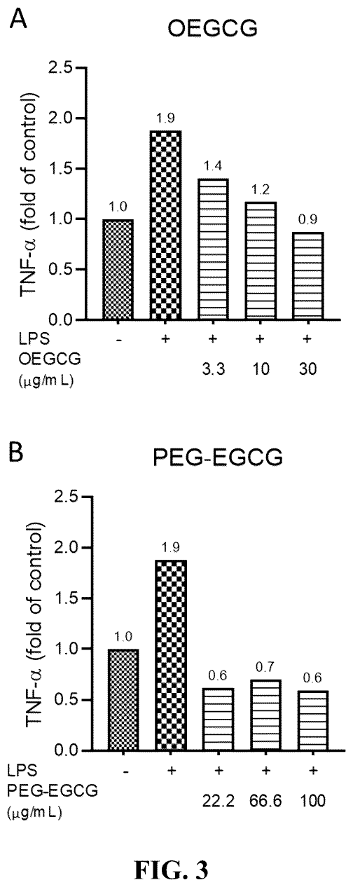

RASF cells were stimulated with LPS and the concentration of TNF-α was elevated. Treatment of OEGCG or PEG-EGCG greatly inhibited TNF-α production in a concentration-dependent manner (FIG. 3). RASF cells can secrete inflammatory cytokine including TNF-α, which promotes immune cell activation and destruction of joints and bones. Reduction of TNF-α by OEGCG and PEG-EGCG treatment have the therapeutic benefit in treating rheumatoid arthritis.

### Example 2: OEGCG and PEG-EGCG Reduce Inflammation in an Inflammatory Bowel Disease Cell Model

### Materials

TRIzol-A+ reagent (Tiagen)

Caco-2 is from ATCC (HTB-37)

### Method

Caco-2 cells were cultured at 37° C. in a humidified chamber of 5% C02 in growth medium. Caco-2 cells were seeded at 2×10⁶ cells/well in 6 well dish overnight and pretreated with OEGCG (3.3 M) or PEG-EGCG (10 M) for 24 hours and then cultured in the presence or absence of LPS (20 g/mL) for another 8 hours. After treatment, total RNA was extracted from the Caco-2 cells using a TRIzol-A+ reagent following manufacturer's instruction. The extracted RNA was reverse transcribed into cDNA according to the manufacturer's protocols (Tiangen, Beijing, China). qRT-PCR was done by QuantStudio™ 6 Flex Real-Time PCR System (Life Technologies, USA) with conditions set to 95° C. for 3 min for the initial denaturation and followed by 35-50 cycles with denaturation at 95° C. for 10 s, annealing at 56° C. for 10 s, and extension at 72° C. for 60 s. The cycle threshold (Ct) values were calculated and normalized to the level of a housekeeping gene GAPDH. The forward and reverse primers were used following the publication from Wu et al. (Inflammation. 2019 December; 42(6):2215-2225.)

### Results

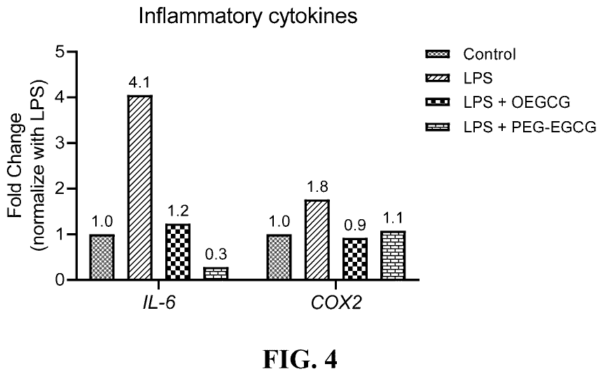

The mRNA level of IL-6 and COX2 in Caco-2 cells were analyzed by qRT-PCR assay. Treatment of OEGCG and PEG-EGCG reduced the mRNA expression of IL-6 and COX2 compared with no treatment group (FIG. 4). These results suggest OEGCG and PEG-EGCG treatment can reduce disease progression via inhibiting inflammatory cytokines production.

### Example 3: MINC-Anti-CD3 Reduces Toxicity of Anti-CD3

### Materials

Anti-CD3 antibody was purchased from Biolegend (Catalog #317325).

MINC-anti-CD3 was prepared according to WO2009/054813.

### Method

Six to eight weeks Balb/c mice were kept in a specific pathogen-free environment. After 3 days acclimation, treatment was started on the day after baseline calculation, designated as day 0. The anti-CD3 or MINC-anti-CD3 were administrated to mice at dose of 5, 25 and 125 g/mouse. i.v. daily for 2 days. The mice were randomly assigned to one of three treatment groups: 1) saline; 2) anti-CD3; 3) MINC-anti-CD3, n=5. Body weight, survival and clinical sign were daily measured every other day for consecutive eight days. Statistical analysis was performed using GraphPad Prizm. Two-Way ANOVA was used to evaluate differences between the groups, with p<0.05 considered significant.

### Results

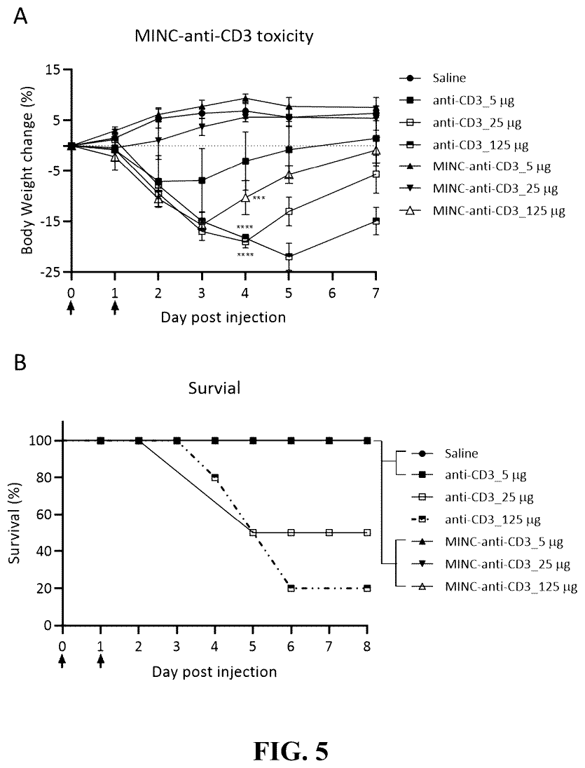

After two shots of treatment, free anti-CD3 antibody significantly reduced mouse weight at day 2 to day 5 (FIG. 5A). Four of five high dose and three of five mid-dose group died through day 5 to day 7 (FIG. 5B). Whereases 125 μg/mouse of MINC-anti-CD3 antibody exhibited moderate weight loss at day 5 and recover at day 7 (FIG. 5A). No animal died in MINC-anti-CD3 groups (FIG. 5B). The result demonstrated MINC encapsulation significantly reduced toxicity and was tolerant to the dose up to 25 folds of clinical dosage (equivalent 5 g/mouse).

Anti-CD3 is a broad-spectrum immune suppressor for treating various autoimmune diseases. However, it's high toxicity restricts its clinical use. This example showed that MINC formulation successfully reduced the toxicity of anti-CD3. MINC-anti-CD3 is a safe drug for the treatment of autoimmune diseases.

### Different Flavonoid Oligomers and Polymer-Flavonoid Conjugates in Forming MINC-Drugs (Examples 4-5)

### Example 4: Method for Preparing MINC-Anti-HER2 Using Different Flavonoids in Flavonoid Oligomers and Polymer-Flavonoid Conjugates

### Material

Anti-HER2 was trastuzumab obtained from Eirgenix.

### Method

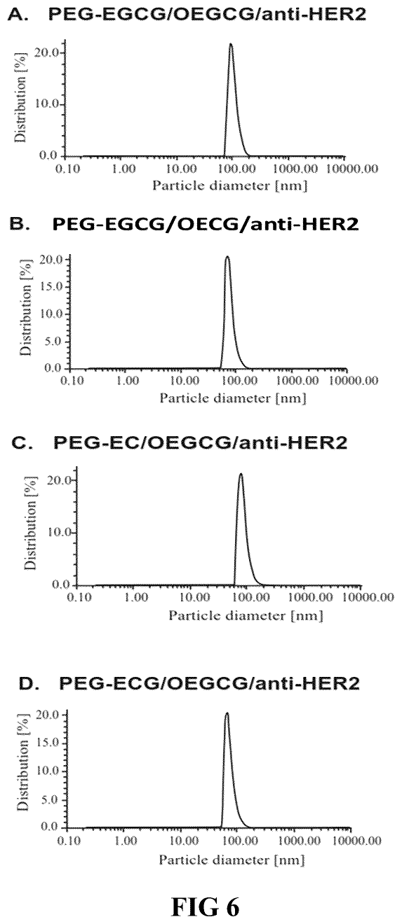

MINC anti-HER2 nanoparticles were prepared according to WO2009/054813. In brief, anti-HER2 was incubated in PBS. Subsequently, different flavonoid oligomer including OEGCG or OECG was added to anti-HER2, followed by adding different polymer-flavonoid including PEG-EGCG, PEG-ECG or PEG-EC. After incubating the mixture at room temperature, 10K MWCO centrifugal filter was used to remove the unreacted oligomer flavonoid and polymer-flavonoid. DLS (Anton Paar Litesizer 500) was used to measure the nanoparticle size and the results are shown in FIG. 6.

### Results

FIG. 6 shows that different polymer-flavonoid conjugates and different flavonoid oligomers all successfully generated MINC-anti-HER2 micelles with particle size around 100 nm. The results demonstrate that homogenous nanoparticles (micelles) were successfully formed with an expected size which is different from unencapsulated anti-HER antibody (about 5-10 nm).

In this example, different flavonoid oligomers used include OEGCG (FIGS. 6A, 6C and 6D) and OECG (FIG. 6B); and different polymer-flavonoids used include PEG-EGCG (FIGS. 6A and 6B), PEG-EC (FIG. 6C) and PEG-ECG (FIG. 6D).

These data support that MINC nanoparticle can be formed by different flavonoid oligomers and different polymer-flavonoid conjugates.

### Example 5: Method for Preparing MINC-BSA Using Different Polymers in Polymer-Flavonoid Conjugates

### Materials

BSA was purchased from Sigma-Aldrich.

### Method

MINC (Multi-target Immune Nanocarrier Combination)-BSA nanoparticles were prepared according to WO2009/054813. In brief, BSA was incubated in PBS. Subsequently, OEGCG was added to BSA, followed by adding different polymer-flavonoid including PEG-EGCG, HA-EGCG and Dextran-EGCG. After incubating the mixture at room temperature, 10K MWCO centrifugal filter was used to remove the unreacted OEGCG and polymer-flavonoid. DLS (Anton Paar Litesizer 500) was used to measure the nanoparticle size.

### Results

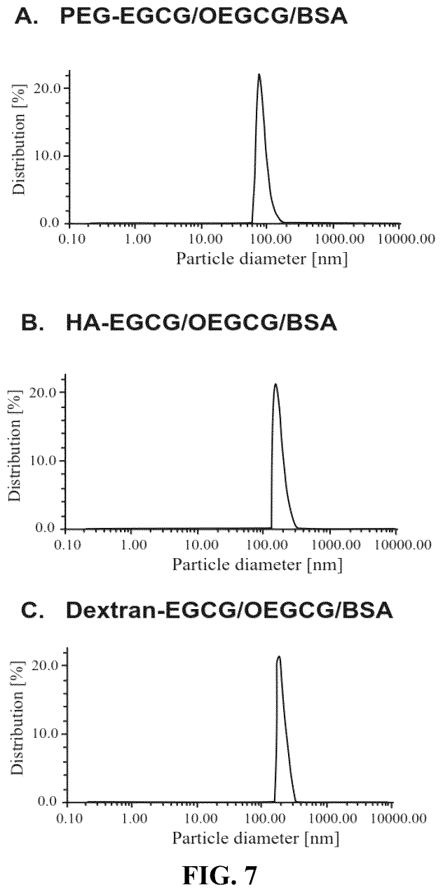

FIG. 7 shows that different polymers in polymer-flavonoid conjugates were used to successfully generate MINC-BSA. The results demonstrate that homogenous nanoparticles (micelles) were successfully formed. These different polymers were PEG (FIG. 7A), HA (FIG. 7B) and dextran (FIG. 7C). Altogether, the data support that MINC nanoparticles could be formed by different polymer-flavonoids conjugates.

### Example 6: MINC-Anti-CD3 Study in Type 1 Diabetes (TID) (Prophetic Example)

### Objective

This experiment is intended to demonstrate that MINC-anti-CD3 treatment can treat T1D by improving blood glucose, insulin sensitivity, HbA1c level and C-peptide level, comparing to saline group. This experiment is intended to show that MINC-anti-CD3 is effective in treating type 1 diabetes.

### Materials

Anti-CD3 antibody is purchased from Biolegend (Catalog #317325).

MINC-anti-CD3 is prepared according to WO2009/054813.

### Method

8 to 15-weeks-old diabetic female NOD mice (NOD/ShiLtJ strain) are monitored twice weekly for diabetes status. Diabetic status is defined as blood glucose ≥200 mg/dL by tail bleed on 2 consecutive days. Mice are assigned to groups including one control group, and treatment groups composed of different dosages of MINC-anti-CD3 or free anti-CD3. The treatment groups are treated with MINC-anti-CD3 or anti-CD3 for 5 consecutive days via i.v. injection, at anti-CD3 equivalents of 1, 5 and 25 μg. Body weight reduction, survival, fasting blood glucose, and C-peptide are measured once per week. HbA1c is measured at day 14, 28 and 42 after first injection. The values of MINC-anti-CD3 and anti-CD3 treatment groups are compared by One-way ANOVA (α=0.05) analysis.

### Example 7. MINC-Anti-CD3 in Treating Rheumatoid Arthritis (RA) (Prophetic Example)

### Material

Rat anti-CD3 antibody is purchased from Biolegend.

MINC-anti-CD3 antibody is prepared according to WO2009/054813.

### Objective

This experiment is intended to demonstrate MINC-anti-CD3 treatment can reduce joint swelling and maintain bone integrity, comparing to saline group. Histopathologic examination is intended to show that MINC-anti-CD3 is better in reducing immune cell infiltration, inflammatory cytokine production and better outcome than saline alone. This experiment is intended to show that MINC-anti-CD3 is effective in treating rheumatoid arthritis.

### Method

Eighty 10-week-old male Wistar rats are purchased from BioLasco. The rats are maintained in a specific pathogen-free facility. In brief, on day 0, 0.2 mL of emulsified bovine type II collagen with Freund's adjuvant is subcutaneously injected into the tail base, followed by a booster injection on day 7. On the same day, rats receive saline or 0.25-6.25 mg/kg of MINC-anti-CD3 via intravenous injection once per week. After administration, rats are examined with bone performance by micro-CT and joint swelling by measuring diameters at the ankle and the hind paw. At the endpoint, rats are sacrificed at day 28 for histopathological examination. The rat ankle joint samples are fixed with 10% formalin. After the decalcification in hydrochloric acid, samples are embedded and fixed in paraffin. The sections are stained with hematoxylin and eosin (H&E) to examine the joint morphology. TNF-α expression level and inflammatory cell infiltration level are examined by anti-TNF-α and anti-CD38 immunohistochemistry staining.

### Example 8. MINC-Anti-TNF-α in Treating Rheumatoid Arthritis (RA) (Prophetic Example)

### Objective

This experiment is intended to demonstrate that MINC-anti-TNF-α treatment can reduce joint swelling and maintain bone integrity, comparing to saline group. Histopathological examination is intended to show that MINC-anti-TNF-α treatment can reduce immune cell infiltration, decrease TNF-α level and have better outcome than saline control. This experiment is intended to show that MINC-anti-TNF-α is effective in treating rheumatoid arthritis.

### Materials

Rat anti-TNF-α antibody is purchased from Biolegend.

MINC-anti-TNF-α antibody is prepared according to WO2009/054813.

### Method

Eighty 10-week-old male Wistar rats are purchased from BioLasco. The rats are maintained in a specific pathogen-free facility. In brief, on day 0, 0.2 mL of emulsified bovine type II collagen with Freund's adjuvant is subcutaneously injected into the tail base, followed by a booster injection on day 7. On the same day, rats receive two shots 2 to 20 mg/kg of MINC-anti-TNF-α via intravenous injection once per week. After administration, rats are examined with bone performance by micro-CT and joint swelling by measuring diameters at the ankle and the hind paw. At the endpoint, rats are sacrificed at day 28 for histopathological examination. The rat ankle joint samples are fixed with 10% formalin. After the decalcification in hydrochloric acid, samples are embedded in paraffin. The sections are stained with hematoxylin and eosin (H&E) to examine the joint morphology. TNF-α expression level and inflammatory cell infiltration level are examined by anti-TNF-α and anti-CD38 immunohistochemistry staining.

### Example 9. MINC-Anti-CD3 in Treating Inflammatory Bowel Disease (IBD) (Prophetic Example)

### Objectives

This experiment is intended to show that DAI scores of rats in the MINC-anti-CD3 treatment group are significantly lower than those in the saline group. Histopathologic examination is intended to show that ulceration with mucosal hyperemia, edema and inflammatory cell infiltration are reduced in the MINC-anti-CD3 treatment group. Overall, this experiment is intended to show that MINC-anti-CD3 has therapeutic efficacy in inflammatory bowel disease.

### Material

Rat anti-CD3 antibody is purchased from Biolegend.

MINC-anti-CD3 antibody is prepared according to WO2009/054813.

### Method

Rats are fasted for 48 hours and anesthetized with 3% sodium pentobarbital. In brief, on day 1, a lavage needle is inserted into the anus and needle is advanced to 8 cm to the anus verge. Next, 1.5-2.0 ml of 8% acetic acid is injected into the colon and add another 3-5 ml saline to spread the acetic acid in the colon. 24 h later, the rats are divided into groups to treat with saline or with 0.25-6.25 mg/kg of MINC-anti-CD3 via i.v. injection on day 2 and day 5. At day 8, rats are sacrificed and the colon and tissue adjacent to the ulcer or hyperemic area are harvested. Fixed and paraffin embedded tissue is stained with hematoxylin and eosin for subsequent histological examination. The disease activity index (DAI) is scored following the criteria below. No weight loss is scored as 0 point, weight loss of 1-5% as 1 point, 5-10% as 2 points, 10-15% as 3 points, and more than 15% as 4 points.

### Example 10 MINC-Anti-TNF-α in Treating Inflammatory Bowel Disease (IBD) (Prophetic Example)

### Objectives

This experiment is intended to show that DAI scores of rats in the MINC-anti-TNF-α treatment group are significantly lower than those in the saline group. Histopathologic examination is intended to show ulceration with mucosal hyperemia, edema and inflammatory cell infiltration are reduced in MINC-anti-TNF-α treatment group. Overall, this experiment is intended to show that MINC-anti-TNF-α has therapeutic efficacy in inflammatory bowel disease.

### Materials

Rat anti-TNF-α antibody is purchased from Biolegend.

MINC-anti-TNF-α antibody is prepared according to WO2009/054813.

### Method

Rats are fasted for 48 hours and anesthetized with 3% sodium pentobarbital. In brief, a lavage needle is inserted into the anus and needle is advanced to 8 cm to the anus verge. Next, 1.5-2.0 ml of 8% acetic acid is injected into the colon and another 3-5 ml saline is added to spread the acetic acid in the colon. 24 h later, the rats are divided into groups to treat with saline or 0.5 to 20 mg/kg of MINC-anti-TNF-α via i.v. injection on day 2 and day 5. At day 8, rats are sacrificed and the colon and tissue adjacent to the ulcer or hyperemic area are harvested. Fixed and paraffin embedded tissue is stained with hematoxylin and eosin for subsequent histological examination. The disease activity index (DAI) following the criteria below. No weight loss is scored as 0 point, weight loss of 1-5% as 1 point, 5-10% as 2 points, 10-15% as 3 points, and more than 15% as 4 points.

### LIST OF ABBREVIATIONS

4-HHR 4-hydroxyhexenal reductase
AChR Acetylcholine receptor
ALOX5 Arachidonate 5-lipoxygenase
ARX Aristaless-related homeobox
ATP4A ATPase H+/K+ transporting alpha subunit
BAFF B-cell activating factor
BTK Bruton's tyrosine kinase
C5 Complement component 5
CCL2 C-C motif chemokine ligand 2
CCR C-C chemokine receptor type
CNTF Ciliary neurotrophic factor
CTLA4 Cytotoxic T-lymphocyte associated protein 4
CX3CL1 CX3C chemokine ligand 1
CXCL10 C-X-C motif chemokine ligand 10
CXCR1/2 C-X-C chemokine receptor type 1/2
DHFR Dihydrofolate reductase
DYRKIA Dual-specificity tyrosine-phosphorylation-regulated kinase 1A
FcRN Neonatal Fc receptor
FLT3L FMS-like tyrosine kinase 3 ligand
FoxP3 Forkhead box P3
GAD Glutamic acid decarboxylase
GCSF Granulocyte colony-stimulating factor
GLP-1 Glucagon-like peptide 1
GM-CSF Granulocyte-macrophage colony-stimulating factor
GRK2 G protein-coupled receptor kinase 2
HDAC Histone deacetylase
HNE 4-Hydroxynonenal
HSP60 Heat shock protein 60
ICAM-1 Intercellular adhesion molecule 1
IFNAR Interferon alpha/beta receptor
IGF Insulin-like growth factor
IGFBP1 Insulin-like growth factor binding protein 1
IL- Interleukin-
iNOS Inducible nitric oxide synthase
IRAK-4 Interleukin-1 receptor-associated kinase 4
ITGA4 Integrin alpha 4
ITGB7 Integrin beta 7
JAK2 Janus kinase 2
JAK3 Janus kinase 3
LRH-1 Liver receptor homolog-1
LypR620W Lymphoid protein tyrosine phosphatase R620W
MDA Malondialdehyde
MDR1 Multidrug resistance protein 1
MEK Mitogen-activated protein kinase kinase
MIP Macrophage Inflammatory Protein
MMP1 Matrix Metalloproteinase 1
NFAT Nuclear Factor of Activated T cells
NF-κB Nuclear Factor kappa B
NIS Sodium/Iodide Symporter
NOX2 NADPH Oxidase 2
NR3C1 Nuclear Receptor Subfamily 3 Group C Member 1 (also known as Glucocorticoid Receptor)
NRF2 Nuclear Factor Erythroid 2-Related Factor 2
p38 MAPK p38 Mitogen-Activated Protein Kinase
PD1 Programmed cell death protein 1
PDE4 Phosphodiesterase 4
PGC-1α Peroxisome Proliferator-Activated Receptor Gamma Coactivator 1-Alpha
PPAR-γ Peroxisome Proliferator-Activated Receptor Gamma
PPAT Phosphoribosyl Pyrophosphate Amidotransferase
PTGS1/2 Prostaglandin-Endoperoxide Synthase 1/2 (also known as COX-1/2)
RAE-1 Retinoic Acid Early Transcript 1
RHOH Ras Homolog Family Member H
RNASET2 Ribonuclease T2
RORgT Retinoic Acid Receptor-Related Orphan Receptor Gamma T
S1P Sphingosine-1-Phosphate
S1PR1 Sphingosine-1-Phosphate Receptor 1
SLAMF6 Signaling Lymphocytic Activation Molecule Family Member 6
STAT1 Signal Transducer and Activator of Transcription 1
TGF-β Transforming Growth Factor Beta
TKI Tyrosine Kinase Inhibitor
TLR9 Toll-Like Receptor 9
TNF Tumor Necrosis Factor
TPO Thrombopoietin
TSHR Thyroid Stimulating Hormone Receptor
TYK2 Tyrosine Kinase 2
VDR Vitamin D Receptor
VMAT-2 Vesicular Monoamine Transporter 2

The invention, and the manner and process of making and using it, are now described in such full, clear, concise, and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as the invention, the following claims conclude this specification.

What is claimed is:

1. A method of treating an autoimmune disease, comprising the step of administering to a subject in need thereof an effective amount of micelles having an outer shell comprising one or more polymer-flavonoid conjugates, optionally an inner shell comprising one or more flavonoid oligomer, and a drug encapsulated within the shells;
wherein the polymer is a hydrophilic polymer having a molecular weight of 1,000 to 100,000 daltons, and is selected from the group consisting of: poly(ethylene glycol) (PEG), hyaluronic acid, dextran, polyethylenimine, poloxamers, povidone, D-alpha-tocopheryl, and polyethylene glycol succinate;
the flavonoid is EGCG, EC, EGC, or ECG, as shown in the structures below: 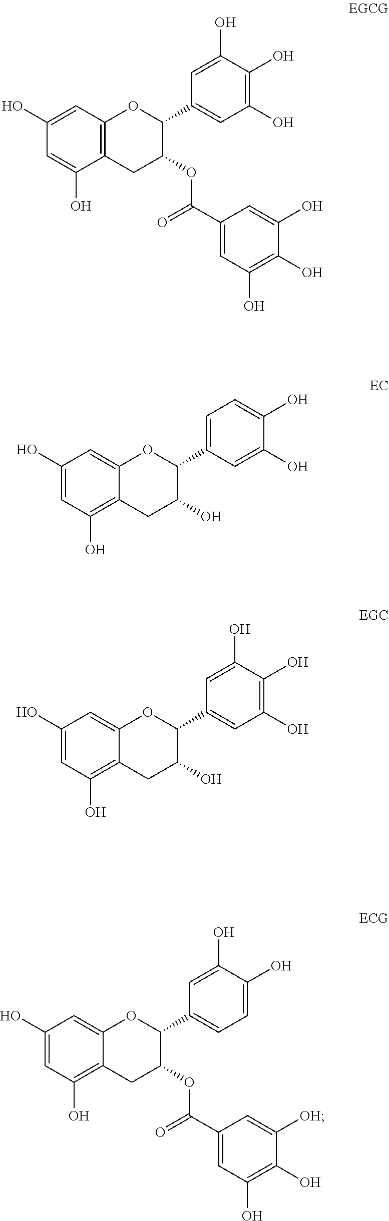 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]
the flavonoid oligomer comprises 2-20 flavonoids of EGCG, EC, EGC, or ECG;
the autoimmune disease is selected from the group consisting of: type 1 diabetes, inflammatory bowel disease, rheumatoid arthritis. multiple sclerosis, systemic lupus erythematosus, psoriasis, myasthenia Gravis, and Hashimoto's thyroiditis.

2. The method according to claim 1, wherein the micelles having an outer shell comprising PEG-EGCG and an inner shell comprising EGCG oligomers.

3. The method according to claim 2, wherein the autoimmune disease is type 1 diabetes, and the drug is anti-CD3, anti-IL-1β, anti-IL-1R, GAD, HSP60, B9-23 peptide, MIP, IL-2, anti-CTLA4, anti-CD40, anti-CD20, anti-PD1, TKI, GLP-1, anti-STAT1, anti-SIP, anti-12/15-LOX, IL-35, HDAC inhibitor, anti-DYRKIA, NFAT, Substance P, LRH-1, IL-10, IGF, IGFBP1, ARX, GCSF, VMAT-2, or RAE-1.

4. The method according to claim 2, wherein the autoimmune disease is inflammatory bowel disease, and the drug is anti-CD3, anti-ITGA4, anti-IL-12B, anti-TNF, anti-JAK2, anti-PTGS1/2, PPAR-γ, anti-ITGB7, NR3C1, anti-JAK3, anti-ALOX5, anti-TYK2, anti-PPAT, VDR, anti-MMP1, anti-MMP7, anti-DHFR, anti-MMP13, anti-ATP4A, S1PR1, anti-IL-36, anti-IL-6, anti-IL-6R, anti-TLR9, IL-10, anti-TGF-β, anti-IL-9, anti-IL-12, anti-smad7, anti-integrins, anti-MDR1, anti-PPAR-7, anti-IL-35, anti-IL-27, or anti-IL17.

5. The method according to claim 2, wherein the autoimmune disease is rheumatoid arthritis, and the drug is anti-TNF-α, anti-IL-1R, anti-IL-1, anti-IL-6R, anti-IL-6, anti-IL-2, IL-10, IL-15, IL-18, anti-IL-17, anti-IL-17R, anti-IFN-γ, anti-CCL2, anti-CCR9, anti-CX3CL1, anti-CCR1, anti-CCR2, anti-CCR5, anti-TLR4, anti-GRK2, anti-MEK, anti-MMP9, anti-CD3, anti-CD80, anti-BTK, anti-IL-23, anti-GM-CSF, anti-CXCL10, anti-CXCL12, anti-CXCL13, anti-CXCL16, anti-CXCR1/2, anti-CXCL3, anti-CXCR4, anti-CXCR7, anti-CCR7, anti-JAK, anti-p38 MAPK, anti-IRAK-4, anti-CD20, anti-CD11a, or anti-CD19.

6. The method according to claim 2, wherein the autoimmune disease is multiple sclerosis, and the drug is ROS scavenger, anti-NF-κB, anti-NOX2, anti-iNOS, MDA, HNE, 4-HHR inhibitor, IFN-β, NRF2 activator, PGC-1α, anti-IL-1β, anti-TNF-α, anti-IL-6, anti-IL-6R, anti-MMP-9, anti-IL-12, anti-IFN-γ, CNTF, anti-caspase 3, anti-MIP-1α, anti-TLR2, anti-CD3, anti-ICAM-1, anti-CCR6, anti-IL2, anti-IL-9, anti-17, TGF-β, IL-4, or FoxP3 activator.

7. The method according to claim 2, wherein the autoimmune disease is systemic lupus erythematosus, and the drug is anti-CD20, anti-CD19, anti-CD22, proteosome inhibitor, anti-BAFF, anti-BTK, ant-CD28, anti-CD40L, anti-CD40, anti-IL12, anti-IL23, anti-IL17, IL2, mTOR inhibitor, calcineurin inhibitor, anti-JAK, anti-IFN-α, anti-IFNAR, anti-IFN-γ, anti-TLR7, anti-TLR9, anti-TLR8, anti-CTLA4, anti-IL6, or anti-CD3.

8. The method according to claim 2, wherein the autoimmune disease is psoriasis, and the drug is anti-CD3, anti-TNF, anti-IL-17A, anti-IL-12, anti-IL-23, anti-IL-36, anti-PDE4, anti-JAK, anti-RORgT, anti-IRAK-4, anti-CCL20, anti-CXCL8, anti-IL-1β, anti-iNOS, anti-IL-8, anti-IFNγ, anti-S1PR1, anti-Tyk2, anti-A3 adenosine receptor, or mTOR inhibitor.

9. The method according to claim 2, wherein the autoimmune disease is Myasthenia Gravis, and the drug is Anti-CD3, Anti-FcRN, anti-CD20, amti-CD19, anti-CD154, proteasome inhibitor, anti-CD38, anti-CD40, anti-IL6, anti-TNF, anti-BARF, anti-BTK, anti-IL-6R, anti-IL-17A, anti-C5, or anti-AChR.

10. The method according to claim 2, wherein the autoimmune disease is Hashimoto's thyroiditis, and the drug is anti-CD3, anti-TSHR, anti-TPO, anti-Tg, anti-NIS, anti-IL-12, anti-IFN-γ, anti-IL-4, anti-IL-5, anti-IL-10, anti-IL-17, anti-IL-6, anti-FLT3L, anti-LypR620W, anti-ICAM1, anti-IL17, anti-IL-23, anti-RHOH, anti-RNASET2, anti-SLAMF6, anti-TNF-α, or anti-FcRN.

11. A method of treating an autoimmune disease, comprising the step of administering to a subject in need thereof an effective amount of one or more polymer-flavonoid conjugates,
wherein the polymer is a hydrophilic polymer having a molecular weight of 1,000 to 100,000 daltons, and is selected from the group consisting of: PEG, hyaluronic acid, dextran, polyethylenimine, poloxamers, povidone, D-alpha-tocopheryl, and polyethylene glycol succinate;
the flavonoid is EGCG, EC, EGC, or ECG, as shown in the structures below: 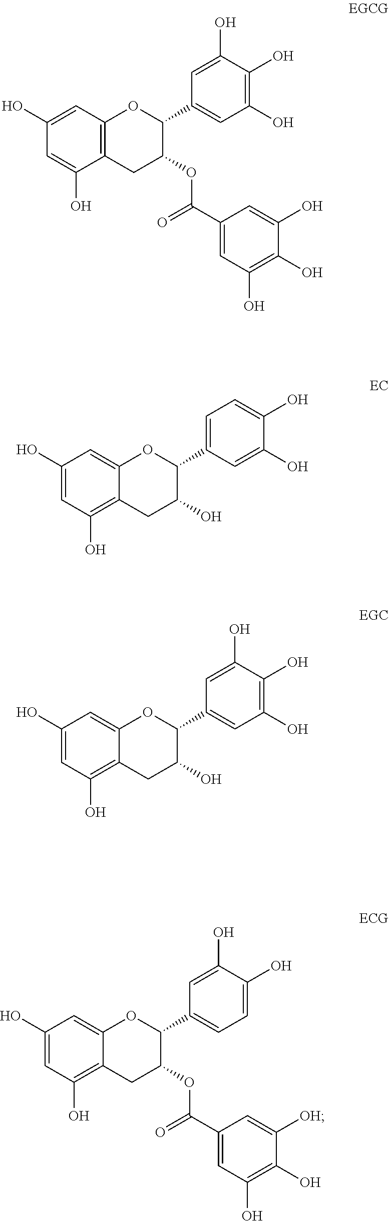 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]
and
the autoimmune disease is selected from the group consisting of: rheumatoid arthritis, psoriatic arthritis, Sjögren's syndrome, systemic lupus erythematosus, inflammatory bowel disease, Celiac disease, ulcerative colitis, type 1 diabetes, Graves' disease, Hashimoto's thyroiditis, Addison's disease, psoriasis, dermatomyositis, Vitiligo, multiple sclerosis, chronic inflammatory demyelinating polyneuropathy, Guillain-Barre syndrome, Myasthenia gravis, autoimmune vasculitis, pernicious anemia, vasculitis, and myositis.

12. The method according to claim 11, wherein the polymer-flavonoid conjugate is PEG-EGCG.

13. The method according to claim 11, wherein the autoimmune disease is associated with joints and muscles and is selected from the group consisting of: rheumatoid arthritis, psoriatic arthritis, Sjögren's syndrome, and systemic lupus erythematosus.

14. The method according to claim 11, wherein the autoimmune disease is associated with digestive tract and is selected from the group consisting of: inflammatory bowel disease, Celiac disease, and ulcerative colitis.

15. The method according to claim 11, wherein the autoimmune disease is associated with endocrine system and is selected from the group consisting of: type 1 diabetes, Graves' disease, Hashimoto's thyroiditis, and Addison's disease.

16. The method according to claim 11, wherein the autoimmune disease is associated with skin and is selected from the group consisting of: psoriasis, dermatomyositis, and Vitiligo.

17. The method according to claim 11, wherein the autoimmune disease is associated with the nervous system and is selected from the group consisting of: multiple sclerosis, chronic inflammatory demyelinating polyneuropathy, and Guillain-Barre syndrome.

18. The method according to claim 11, wherein the autoimmune disease is Myasthenia gravis, autoimmune vasculitis, pernicious anemia, vasculitis, or myositis.

19. A method of treating an autoimmune disease, comprising the step of administering to a subject in need thereof an effective amount of one or more flavonoid oligomers,
wherein the flavonoid is EGCG, EC, EGC, or ECG, as shown in the structures below: 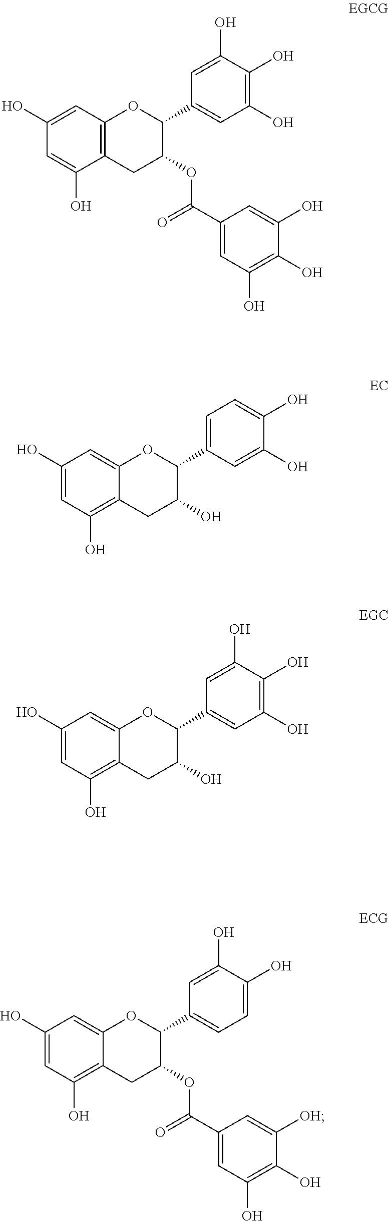 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]
the flavonoid oligomer comprises 4-12 flavonoids of EGCG, EC, EGC, or ECG; and
the autoimmune disease is selected from the group consisting of: rheumatoid arthritis, psoriatic arthritis, Sjögren's syndrome, systemic lupus erythematosus, inflammatory bowel disease, Celiac disease, ulcerative colitis, type 1 diabetes, Graves' disease, Hashimoto's thyroiditis, Addison's disease, psoriasis, dermatomyositis, Vitiligo, multiple sclerosis, chronic inflammatory demyelinating polyneuropathy, Guillain-Barre syndrome, Myasthenia gravis, autoimmune vasculitis, pernicious anemia, vasculitis, and myositis.

20. The method according to claim 19, wherein the flavonoid oligomer is oligomer of EGCG.
